(19) United States
(12) Patent Application Publication
(10) Pub. No.: US 2011/0171643 A1
(43) Pub. Date: Jul. 14, 2011

(54) METHOD, PROCESS, AND KIT FOR CANCER DIAGNOSIS, PROGNOSIS, AND AFTERCARE

(75) Inventors: Guorong LI, Saint-Etienne (FR); Jacques Tostain, L'Etrat (FR); Christian Genin, Saint Etienne (FR)

(73) Assignees: Chu Saint Etienne Chu-Hopital Nord, Saint Priest en Jarez (FR); Universite Jean Monnet, Saint Etienne Cedex (FR)

(21) Appl. No.: 12/920,165

(22) PCT Filed: Mar. 9, 2009
(86) PCT No.: PCT/FR2009/050381

§ 371 Date: Dec. 3, 2010

(30) Foreign Application Priority Data
Mar. 14, 2008 (FR) 0801401

(51) Int. Cl. C12Q 1/68; C07H 21/04

(52) U.S. Cl. 435/6.11; 435/6.1; 435/6.12; 536/24.33; 536/24.31

(57) ABSTRACT

A method or process for the diagnosis/prognosis/follow-up of cancer in vertebrates, comprising the following steps:
i) extracting nucleic material from a biological sample;
ii) using at least one pair of amplification primers in order to obtain amplicons of at least one target sequence;
iii) using at least one detection probe for detecting the presence of the said amplicons;
   characterised in that the extraction step is carried out from extracellular nucleic material from a biological sample. More precisely, the said nucleic material is RNA or DNA of the CA9 gene circulating in the free state in a biological sample.

The invention also relates to the pairs of amplification primers and/or a detection probe for the diagnosis/prognosis/follow-up of cancer including at least 15 nucleotide patterns of a nucleotide sequence selected from the SEQ ID nos. 1 to 6, and a kit including the said at least one pair of primers and/or at least the said probe.

This invention relates to a method, process and kit for the early detection of cancer for the purpose of diagnosis, prognosis or therapeutic follow-up.

More particularly, this invention relates to a new method, amplification primers and hybridisation probes that may be used as part of the method, and a kit for the diagnosis/prognosis/follow-up of cancer in vertebrates.

“Diagnosis” means the determination of the affection of a person suffering from a given disorder; “prognosis” means the degree of seriousness indicative of the subsequent development of a disorder and “therapeutic” refers to the curative or palliative treatment offered to an individual.

Most often, cancer of the kidney, for example, is asymptomatic and the diagnosis is made through radiological examinations such as ultrasound scans, tomodensitometry (CT) scans, urography or magnetic resonance imagery (MRI) etc.

However, in the very early stages, small tumours are difficult to detect and it is not easy to determine their cancerous nature through imagery. Repeat imagery examinations and/or a histological or cytological test after a biopsy or puncture are required.

These examinations are expensive, not sufficiently sensitive and some biological sampling procedures are very invasive and/or painful.

Some molecular biology tests are focussed on the search for messenger RNA (mRNA) or assays of the protein of an MN/CA9 gene present in the malignant cells of individuals with cancer.

MN/CA9 is an effective molecular marker for detecting cancerous cells. It is also called carbonic anhydrase 9 or CA9. This gene encodes an oncoprotein that is an MN membrane antigen, the isoenzyme MN/CA9 (gene bank accession number NM 000009). The main function of the CA9 gene is pH regulation and it is expressed in many cancers, particularly cancers of the kidney, particularly in the most frequent type of tumour, the conventional cell renal carcinoma (CCRC).

Current research attempts to detect and quantify the mRNA of the genes involved in cancers in circulating tumoral cells from cell extracts or the presence of the CA9 protein in a biological sample.

However, these studies are limited to either the samples taken from a tumour or very low sensitivity in the blood of cancer patients. These tests are thus not always relevant as diagnosis tests.

Further, the tests do not allow screening in early stages at the time of initial diagnosis or relapse.

US patent application US2007224606 relates to a prognosis method comprising the detection of the presence of expression products of the CA9 gene. It discloses a correlation between the expression of the CA9 gene and the prognosis of cancer of the kidney. However, the threshold value used is 50% of positive cells in a tumour sample.

International patent application WO0110910 shows a method that uses the expression protein of the CA9 gene as a marker in order to improve cytological diagnosis.

The method according to this invention particularly makes it possible to determine whether or not cancer is present, asses its seriousness, identify the most suitable treatment and follow up treated patients.

This invention makes up for the drawbacks of the earlier art by offering a new method, process and kit for detecting cancer for the purpose of diagnosis, prognosis or therapeutic follow-up.

The method according to the invention is more specific and much more sensitive.

Additionally, the method according to the invention is simple and reproducible, while being less expensive.

The process according to the invention is also non invasive and not painful.

This invention is aimed at remedying the drawbacks mentioned above, and consists, to that end, in a process or method for diagnosis/prognosis/follow-up of cancer in vertebrates comprising the following steps:
i) extracting nucleic material from a biological sample;
ii) using at least one pair of amplification primers in order to obtain amplicons of at least one target sequence;
iii) using at least one detection probe to detect the presence of the said amplicons;
   characterised in that the extraction step is carried out using extracellular nucleic material from a biological sample.

More precisely, the said nucleic material is RNA or DNA from the CA9 gene circulating in the free state in a biological sample.

Advantageously, when the said nucleic material is RNA, it undergoes reverse transcription. Also, in that case, the reverse transcription step and step (ii) are carried out at the same time (RT-PCR).

The said step (ii) according to the invention is conventional or quantitative PCR.

Advantageously, steps (ii) and (iii) of the method according to the invention are carried out at the same time.

The said detection probe may also include a marker.

More precisely, the said pair of primers and/or the said detection probe include at least 15 nucleotide patterns of a nucleotide sequence of the CA9 gene selected from SEQ ID nos. 1 to 6.

This invention also relates to:
at least one amplification primer for diagnosis/prognosis/follow-up of cancer including at least 15 nucleotide patterns of a nucleotide sequence selected from SEQ ID nos. 1 to 6.
a detection probe for the diagnosis/prognosis/follow up of cancer including at least 15 nucleotide patterns of a nucleotide sequence selected from SEQ ID nos. 1 to 6.

Advantageously, the invention also relates to a kit for the diagnosis/prognosis/follow-up of cancer comprising at least one pair of the primers mentioned above and/or at least one detection probe mentioned above.

The invention will be better understood in the light of the description below, relating to illustrative examples, that are not exhaustive in any way, of this invention, by reference to the enclosed drawings, wherein:

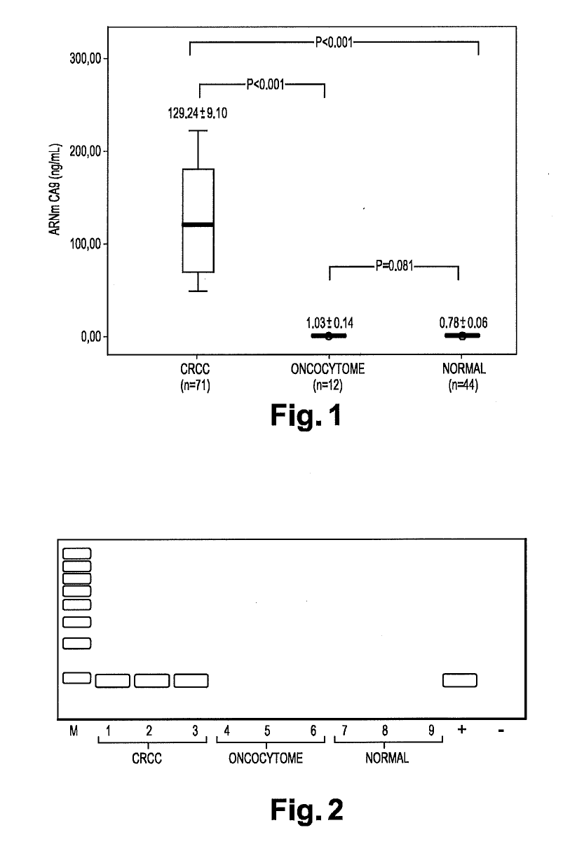

FIG. 1 illustrates the CA9 mRNA rates of different individuals;

FIG. 2 is an electrophoresis gel showing the results of a conventional PCR;

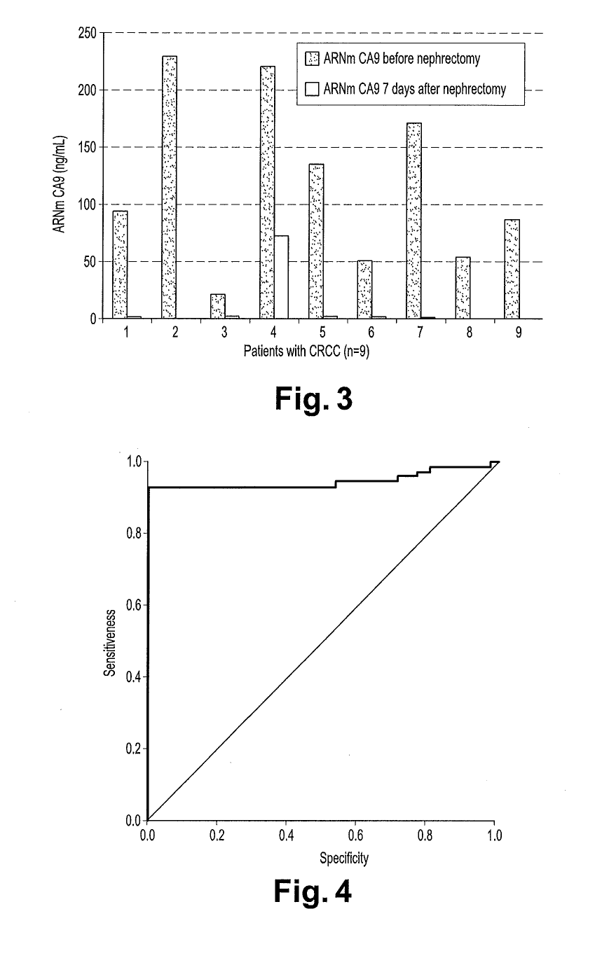

FIG. 3 represents the pre and post-operation rates of CA9 mRNA in individuals with CCRC;

FIG. 4 is a ROC curve that demonstrates the diagnostic utility of the CA9 mRNA rate.

This invention offers a method, process, test and kit for detecting cancer for the purpose of diagnosis, prognosis or therapeutic follow-up, based on the detection of a specific tumoral biomarker in material taken from a biological sample.

A “biological sample” is taken from an individual for the purpose of screening/diagnosis/follow-up of cancer, particularly of the kidney. That sample, which contains cells and nucleic material, may be the individual's blood, stools, saliva, urine, bile, serum, plasma, semen etc. or a tumour sample.

The nucleic material made up of sequences of deoxyribonucleic acid (DNA) or ribonucleic acid (RNA), taken in that manner from the said individual through the said biological sample includes a target sequence. A “target sequence” is a specific nucleotide sequence of a target gene, whether single or double-stranded, such as the CA9 gene.

A “nucleotide sequence” is a sequence of nucleotide patterns, i.e. a sequence of nucleic acids or polynucleotides or fragments thereof.

A “nucleotide pattern” is a natural nucleotide of nucleic acid or a modified nucleotide (base, phosphate, sugar).

The structures and modifications of these sequences are either natural or the result of genetic recombination or chemical synthesis.

The present inventors have focused their search on assays of nucleic acids of a specific gene that circulates in the free state in a biological sample, in order to create a biomarker for the detection of cancer.

For example, the presence of tumoral RNA or DNA directly in a biological sample, such as an analysis of the mRNA of a gene in the serum of a vertebrate.

Indeed, as an illustration, they have been able to show that the mRNA of the CA9 gene could be detected directly in the serum or plasma of an individual with cancer of the kidney.

FIG. 1 shows the rate of CA9 mRNA in individuals with cancer of the kidney (CCRC), benign tumours (oncocytoma) and in healthy individuals. The chart shows that the rates of mRNA of the CA9 gene in individuals with CCRC are significantly higher than in healthy individuals or individuals with benign tumours.

Until now, nucleic acids such as extracellular DNA or RNA were considered to be too fragile and rapidly destroyed by enzymes or by freezing. That meant that they could not withstand such conditions, which major prejudice has now been overcome.

The present inventors have also been able to demonstrate the presence of nucleic acids of the MN/CA9 gene in samples of freshly collected serum, and also after simple coagulation of the blood or after they are frozen.

The method according to the invention relates to the use of a new molecular marker, the CA9 gene, particularly its extracellular circulating nucleic acids in a biological sample.

More precisely, the extracellular mRNA or DNA of the CA9 gene or that which is circulating in the free state is searched by extracting the RNA or DNA and then carrying out an amplification step (conventional or quantitative PCR) of a biological sample.

Other methods may be used to quantify the mRNA or DNA of the CA9 gene.

Further, the method according to the invention also relates to new nucleotide sequences of a target sequence, particularly those of the CA9 gene, which may be used as amplification primers or hybridisation probes for the purposes of detection and/or prognosis and/or follow up of individuals suffering from cancer.

More particularly, the inventors have discovered that the said extracellular target nucleotide sequences were present in biological samples taken in a non-invasive manner from individuals with cancer, particularly of the renal type.

More precisely, the said nucleotide sequence according to the invention including at least 15 nucleotide patterns of a sequence selected from SEQ ID nos. 1 to 6 is very relevant:
as an amplification primer for amplifying target sequences such as the CA9 gene,
as a hybridisation probe for specific hybridisation with target sequences such as the CA9 gene.

According to this invention, an “amplification primer” is a nucleic sequence including 15 to 200 nucleotide patterns, preferably 15 to 25 base pairs of at least one target sequence of genetic material.

“Hybridisation” is a process by which two nucleic sequences, such as for instance a primer and a target sequence, are linked in specific conditions that are well known to those skilled in the art.

A “hybridisation probe” is a nucleic sequence of 15 to 200 nucleotide patterns, preferably 100 to 190 base pairs of at least one target sequence of genetic material. The probe has hybridisation specificity in specific and determined conditions so that it hybridises with the target nucleic sequence.

The sequence of the mRNA of the CA9 target gene is (Genebank accession number NM 001216):

gcc cgt aca cac cgt gtg ctg gga cac ccc aca gtc
agc cgc atg gct ccc ctg tgc ccc agc ccc tgg ctc
cct ctg ttg atc ccg gcc cct gct cca ggc ctc act gt
g caa ctg ctg ctg tca ctg ctg ctt ctg atg cct gtc
cat ccc cag agg ttg ccc cgg atg cag gag gat tcc
ccc ttg gga gga ggc tct tct ggg gaa gat gac cca
ctg ggc gag gag gat ctg ccc agt gaa gag gat tca
ccc aga gag gag gat cca ccc gga gag gag gat cta
cct gga gag gag gat cta cct gga gag gag gat cta
cct gaa gtt aag cct aaa tca gaa gaa gag ggc tcc
ctg aag tta gag gat cta cct act gtt gag gct cct
gga gat cct caa gaa ccc cag aat aat gcc cac agg
gac aa a gaa ggg gat gac cag agt cat tgg cgc tat
gga ggc gac ccg ccc tgg ccc cgg gtg tcc cca gcc
tgc gcg ggc cgc ttc cag tcc ccg gtg gat atc cgc
ccc cag ctc gcc gcc ttc tgc ccg gcc ctg cgc ccc
ctg gaa ctc ctg ggc ttc cag ctc ccg ccg ctc cca
gaa ctg cgc ctg cgc aac aat ggc cac agt gtg caa
ctg acc ctg cct cct ggg cta gag atg gct ctg ggt
ccc ggg cgg gagt acc ggg ctc tgc agc tgc atc tgc
act ggg ggg ctg cag gtc gtcc ggg ctc gga gca cact
gtg gaa ggc cac cgt ttc cct gcc gag atc cac gtg
gtt cac ctc agc acc gcc ttt gcc aga gtt gac gag
gcc ttg ggg cgc ccg gga ggc ctg gcc gtg ttg gcc
gcc ttt ctg gag gag ggc cc g gaa gaa aac agt gcc
tat gag cag ttg ctg tct cgc ttg gaa gaa atc gct
gag gaa ggc tca gag act cag gtc cca gga ctg gac
ata tct gca ctc ctg ccc tct gac ttc agc cgc tac
ttc caa tat gag ggg tct ctg act aca ccg ccc tgt
gcc cag ggt gtc atc tgg act gtg ttt aac cag aca
gtg atg ctg agt gct aag cag ctc cac acc ctc tct
gac acc ctg tgg gga cct ggt gac tct cgg cta cag
ctg aac ttc cga gcg acg cag cct ttg aat ggg cga
gtg att gag gcc tcc ttc cct gct gga gtg gac agc
agt cct cgg gct gct gag cca gtc cag ctg aat tcc
tgc ctg gct gct ggt gac atc cta gcc ctg gtt ttt
ggc ct cct ttt tgc tgt cac cag cgt cgc gtt cct
tgt gca gat gag a ag gca gca cag aag ggg aac caa
agg ggg tgt gag cta ccg ccc agc aga ggt agc cga
gac tgg agc cta gag gct gga tct tgg aga atg tga
gaa gcc agc cag agg cat ctg agg ggg agc cgg taa
ctg tcc tgt cct gct cat tat gcc act tcc ttt taa
ctg cca aga aat ttt tta aaa taa ata ttt ata at

i) The First Step Consists in Extracting Nucleic Material from a Biological Sample.

The detection of the presence of tumoral RNA or DNA from a biological sample requires the extraction of total RNA or DNA from the said sample.

Such extraction is carried out by any protocol for the extraction of nucleic acid from biological samples of a type known in itself. This step of purification consists in separating the nucleic acid from the other constituents and concentrating it.

For example, the biological sample may be blood (5 ml), which is then centrifuged at 1200 g for 10 minutes at 4° C. In that way, the serum is separated before the total RNA is extracted.

The extraction of total RNA may be carried out, for instance, using the RNeasy kit from Qiagen in accordance with the manufacturer's recommendations.

The RNA samples are then stored at −80° C.

Of course, those skilled in the art know how to adapt the extraction, purification and preservation of nucleic acids depending on the biological samples from which they are derived.

As the object of the search is nucleic acids circulating in the free state, that is extracellular nucleic acids, it is not necessary to lyse the circulating tumoral cells. That results in a simpler process that is less time consuming and is less expensive.

### If an RNA Assay is Required, the Step (i) B Consists in Reverse Transcription.

The reverse transcription step consists in synthesising a strand of complementary DNA (cDNA) from mRNA. The synthesis of double-strand cDNA that results from subsequent amplification makes it possible to clone the required genes so as to use them as nucleotide probes for the detection or assay of corresponding mRNA.

After purification, the mRNA undergoes reverse transcription into single-strand cDNA using an enzyme with reverse transcriptase activity. This step may be completed using commercially available kits such as the SuperScript™ First-Strand Synthesis System for RT-PCR kit marketed by Invitrogen or using any method known in itself.

ii) Step 2 is PCR (Polymerase Chain Reaction) Amplification of the DNA or cDNA.

The process of amplification by enzymatic polymerisation (targeted in vitro replication technique called “PCR” or Polymerase Chain Reaction) makes it possible to obtain, from a sample containing DNA or cDNA, important quantities of a specific DNA fragment, such as a tumoral marker, with a definite length by using a pair of nucleotide primers.

This step is carried out with the help of amplification primers in order to generate amplicons of at least one target sequence of the nucleic material.

The said primer and/or said probe include at least 15 nucleotide patterns from a sequence selected from:
a sequence from SEQ ID nos. 1 to 6;
a homologous sequence of SEQ ID nos. 1 to 6 complementary or sufficiently complementary; or sufficiently homologous to hybridise with SEQ ID nos. 1 to 6 or their complementary sequences;
a sequence including a sequence from SEQ ID nos. 1 to 6, which would have the same function as the amplification primer.

The primer sequences and/or probes including at least 15 nucleotide patterns of a nucleotide sequence of the CA9 gene are selected from:

(SEQ ID no. 1)
GGA CAA AGA AGG GGA TGA CC
(SEQ ID no. 2)
AGT TCT GGG AGC GGC GGG A
(SEQ ID no. 3)
CAG AGT CAT TGG CGC TAT GGA
(SEQ ID no. 4)
CGA GCT GGG GGC GGA TA
(SEQ ID no. 5)
gga caa aga agg gga tga cca gag tca ttg gcg cta
tgg agg cga c cc gcc ctg gcc ccg ggt gtc ccc agc
ctg cgc ggg ccg ctt cca gtc ccc ggt gga tat ccg
ccc cca gct cgc cgc ctt ctg ccc ggc cct gcg ccc cc
t gga act cct ggg ctt cca gct ccc gcc gct ccc aga
act
(SEQ ID no. 6)
cag agt cat tgg cgc tat gga ggc gac ccg ccc tgg
ccc cgg gtg tcc cca gcc tgc gcg ggc cgc ttc cag
tcc ccg gtg gat atc cgc ccc cag ctc g

These primers are designed so as to overlap the splice junction in order to eliminate the signals generated by genome contamination.

As an illustration, conventional PCR may be carried out with the Eppendorf MasterMix kit according to the manufacturer's recommendations or using any method known in itself.

For example, the primers used from the CA9 target sequence (186 bp) are SEQ ID nos. 1 and 2.

The following PCR cycle may be used to carry out the gene amplification of any specific sequence using a thermal cycler (for example Perkin Elmer).

After a preliminary denaturation step at 94″C (2 minutes), thirty-five PCR cycles may thus be carried out in order to sufficiently amplify the genetic material. Each cycle may include a new denaturation step at 94° C. (1 minute) followed by a step for the so-called hybridisation of primers at 57° C. (1 minute) and lastly a last step for so-called elongation at 72° C. (1 minute for 1000 nucleotides to amplify). One final step at 72° C. (5 minutes) is also necessary in order to complete the elongation of all the fragments. The primer hybridisation temperature must be calculated depending on the Tm of each primer.

Alternatively, when the mRNA from a biological sample is to be analysed, reverse transcription and PCR (RT-PCR) are carried out simultaneously in one step (steps i-b and ii at the same time). One-step RT-PCR may be carried out using, for example, the kits Super Script™ One-Step RT-PCR and Platinum tag from Invitrogen in accordance with the manufacturer's recommendations. The RT-PCR reaction will also be carried out with a thermal cycler (for example Perkin Elmer) in the following conditions—one step at 50° C. (30 min) allowing reverse transcription followed by a denaturation step at 94° C. (2 min), then followed by 35 cycles of PCR as described above.

For each PCR test, negative controls (with no nucleic acid) and positive controls (for example from a plasmid encoding the CA9 gene) are carried out in parallel.

Advantageously, the PCR may be quantitative. In this way, the SYBR green technique is used, based on the standard curve obtained from plasmids encoding the CA9 gene.

In that case, the primers of the CA9 target sequence (100 bp) may be SEQ ID nos. 3 and 4.

Quantitative PCR is carried out after the RT-PCR reaction described above with the help of the QuantiTect SYBR Green PCR Master Mix kit from Qiagen in accordance with the manufacturer's recommendations. The conditions of the PCR reaction carried out with a thermal cycler (for example Perkin Elmer) are as follows: a denaturation step at 95° C. (10 minutes), followed by forty cycles made up of a new denaturation step at 95° C. (15 seconds), a hybridisation step at 58° C. (15 seconds) and an elongation step at 72° C. (30 seconds). A final cycle made up of a denaturation step at 95° C. (15 seconds), a final elongation step at 60° C. (1 minute) and a last denaturation step at 95° C. (15 seconds) must be carried out in accordance with the manufacturer's recommendations.

iii) Step 3 is Detection.

During the target nucleic acid detection step, use may be made of a specific detection probe.

Of course, this direct or indirect detection step may be carried out using any other mode of detection of a type known in itself.

Advantageously, the hybridisation probe is a detection probe. The hybridisation probe may include a marker that allows it to be detected. The said “marker” is a tracer capable of locating, marking or differentiating what is recognisable because of its physical or chemical properties (radioactivity, fluorescence, mass, colour, luminescence etc. via optical, electrical and other detection methods) in very small quantities.

Functional primers may be analysed with a fluorochrome or another fluorescent or quencher that links specifically with the amplification product (double-strand DNA).

In that way, a hybridisation reaction between a detection probe and the target sequence can be detected.

Alternatively, the detection probe may be a molecular beacon detection probe.

The hybridisation probe may also be a capture probe, where the said probe is immobilised on a solid substrate by any appropriate means (of a type known in itself). After that, the hybridisation reaction between the capture probe and the target sequence is detected.

For detecting the hybridisation reaction, use may also be made of marked target sequences, directly or indirectly from the target sequence.

Alternatively, the hybridisation step may be a marking and/or splitting step of the target sequence.

In order to do away with the primer dimers that are constituted spontaneously, a specific TaqMan probe built with the help of well known software may be linked to the sense and antisense primers.

In order to correct any possible variability of the enzymatic efficiency, the present inventors have standardised the expression of the target gene by determining a ratio between the target gene and a housekeeping gene (NADPH or β-actin for example), the expression of which is required and common in all individuals. The primers of housekeeping genes and particularly NADPH are for instance:

| | |
|---|---|
| sense primer | 5′ AAA GGA CAT TTC CAC CGC AAA 3′ |
| antisense primer | 5′ GGT CGG GTC MC GCT AGG CT 3′ |

Alternatively, step (ii), (conventional or quantitative PCR) is carried out at the same time as the detection step, step (iii).

For example, the products of PCR or amplicons may be separated by electrophoresis on 1.0% agarose gel, and then seen by illumination under UV after staining the DNA with ethidium bromide.

The expected fragment is identified by the comigration of a molecular size marker.

FIG. 2 thus shows the positivity of CA9 mRNA in the case of CCRC and negativity with benign tumours (oncocytoma) and in healthy individuals. M means a molecular size marker.

The results explained below are illustrative of comparison experiments carried out and are not limitative in any case.

TABLE 1
Table 1: Characteristics of individuals

| | | Number of |
|---|---|---|
| | | cases (%) |
| Patients with CRCC (N = 71) | | |
| Gender | | |
| Male | 45 | 63.4% |
| Female | 26 | 36.6% |
| Age (years) | | |
| Range | 35-86 | |
| MedianAverage | 66 | |
| TNM | | |
| T1 | 30 | 42.3% |
| T2 | 5 | 7.0% |
| T3 | 36 | 50.7% |
| Grade | | |
| G1 | 12 | 16.9% |
| G2 | 32 | 45.1% |
| G4 | 5 | 7.0% |
| G3 | 22 | 31.0% |
| G4 | 5 | 7.0% |
| Patients with Oncocytome (N = 12) | | |
| Gender | | |
| Male | 4 | 33.4% |
| Female | 8 | 66.6% |
| Age (years) | | |
| Range | 27-76 | |
| Median | 58 | |
| Individual without tumours (N = 44) | | |
| Gender | | |
| Male | 36 | 81.8% |
| Female | 8 | 18.2% |
| Age (years) | | |
| Range | 50-80 | |
| Median | 64 | |

TABLE 2
Table 2: Quantitative PCR of the various groups

| | Number of cases | CA9 (ng/mL) | P- value |
|---|---|---|---|
| Patients | | | |
| Age | | | 0.858 |
| ≦50 | 11 | 123.32 ± 25.64 | |
| 51-69 | 33 | 130.79 ± 11.95 | |
| ≧70 | 27 | 129.81 ± 16.36 | |
| Gender | | | 0.535 |
| Male | 45 | 131.99 ± 11.03 | |
| Female | 26 | 124.47 ± 16.16 | |
| TNM | | | 0.040 |
| T1 | 30 | 137.53 ± 14.03 | |
| T2 | 5 | 113.23 ± 22.42 | |
| T3 | 36 | 194.81 ± 12.53 | |
| Grade | | | 0.691 |
| 1 | 12 | 154.02 ± 26.68 | |
| 2 | 32 | 123.22 ± 13.18 | |
| 3 | 22 | 118.47 ± 13.39 | |
| 4 | 5 | 155.71 ± 48.91 | |
| Tumour size | | | 0.445 |
| <4 cm | 23 | 131.65 ± 15.83 | |
| 4-8 cm | 32 | 136.24 ± 13.83 | |
| >8 cm | 16 | 111.80 ± 19.22 | |
| Patients with Oncocytome | | | |
| Gender | | | 0.416 |
| Male | 4 | 1.18 ± 0.15 | |
| Female | 8 | 0.96 ± 0.29 | |
| Individuals without tumour | | | |
| Gender | | | 0.540 |
| Male | 36 | 0.81 ± 0.07 | |
| Female | 8 | 0.87 ± 0.17 | |

Table 1 above describes the characteristics of the individuals in the study, as an example, for this invention: 71 individuals with CCRC, 12 individuals with renal oncocytoma and 44 healthy individuals.

Table 2 above provides the results of quantitative PCR in the different groups. The results indicate that the CA9 mRNA rates are not linked to the size of the tumour.

### Table 3: COMPARISON of Tests According to the Invention and ELISA (Human Carbonic Anhydrase IX; R&D Systems):

| | RT-PCR in one step | PCR Quantitative | ELISA |
|---|---|---|---|
| Sensitiveness | 93.0% | 91.5% | 34.8% |
| Specificity | 100.0% | 100.0% | 89.6% |
| Predictive positive | 100.0% | 100.0% | 86.5% |
| value | | | |
| Predictive negative | 89.8% | 88.0% | 41.7% |
| value | | | |

Table 3 above compares the results of the test according to the invention for detecting gene CA9 mRNA in serum/plasma with that of the ELISA test using the CA9 protein in serum for diagnosing cancer.

To that end, a positivity limit of the ELISA technique was first calculated over a population of 48 healthy individuals. That positivity limit is 100 pg/ml and is classically equal to the average of the samples plus or minus two standard deviations (+/−2SD). The results of the technique for detecting CA9 mRNA are much better that those of the ELISA test in terms of sensitivity, specificity and predictive value.

FIG. 3 shows the decrease in the rate of CA9 mRNA after surgery (nephrectomy). Indeed, CA9 mRNA rates decrease significantly or disappear a week after the tumour is removed.

FIG. 4 is the ROC (Receiver Operating Characteristic) curve that demonstrates the diagnosis usefulness of the CA9 mRNA rate. The curve is used to analyse the changes to the specificity and sensitivity of a test with different discrimination threshold values. The area under the ROC curve is an estimator of the overall efficiency. In this case according to the invention, the area under the curve is 0.946 (confidence interval 95%: 0.904-0.993) meaning that the test is perfectly discriminatory.

As compared to the tests currently available in the market, the test according to this invention differs in the use of a new molecular marker and particularly the extracellular nucleic acids of the CA9 gene, with a technique for amplification and/or detection and/or quantification (conventional or quantitative PCR or RT PCR), and a non-invasive method, while providing a good cost to efficiency ratio.

Of course, the method and the test according to the invention may be combined with or include other molecular markers in order to further increase its sensitivity and specificity depending on the cancer to be searched.

This invention further makes it possible to improve the screening strategy and the treatment of early forms of cancer or precancerous conditions.

1. A method or process for the diagnosis/prognosis/follow-up of cancer in vertebrates, comprising the following steps:
i) extracting extracellular nucleic material from a biological sample;
ii) using at least one pair of amplification primers in order to obtain amplicons of at least one target sequence;
iii) using at least one detection probe to detect the presence of the said amplicons;
wherein the said nucleic material is RNA or DNA of the CA9 gene circulating in the free state in the said biological sample.

2. A method according to claim 1, wherein when the said nucleic material is RNA, it undergoes reverse transcription.

3. A method according to claim 2, wherein the reverse transcription step and step (ii) are carried out at the same time (RT PCR).

4. A method according to claim 1, wherein the said step (ii) is conventional or quantitative PCR.

5. A method according to claim 1, wherein the steps (ii) and (iii) are carried out at the same time.

6. A method according to claim 1, wherein the said detection probe includes a marker.

7. A method according to claim 1, wherein the said pair of primers and/or the said detection probe include at least 15 nucleotide patterns of a nucleotide sequence of the CA9 gene selected from SEQ ID nos. 1 to 6.

8. An amplification primer of an extracellular DNA or RNA sequence of the CA9 gene circulating in the free state in a biological sample, for the diagnosis/prognosis/follow-up of cancer, including at least 15 nucleotide patterns of a nucleotide sequence selected from SEQ ID nos. 1 to 6.

9. A probe for detecting an extracellular DNA or RNA sequence of the CA9 gene circulating in the free state in a biological sample, for the diagnosis/prognosis/follow up of cancer, including at least 15 nucleotide patterns of a nucleotide sequence selected from SEQ ID nos. 1 to 6.

10. A kit for the diagnosis/prognosis/follow-up of cancer including at least one pair of primers according to claim 8.

11. A kit for the diagnosis/prognosis/follow-up of cancer including at least one detection probe according to claim 9.


SEQUENCE LISTING (6 sequences)

SEQ ID NO: 1 - DNA, 20 bp, Homo sapiens
ggacaaagaaggggatgacc

SEQ ID NO: 2 - DNA, 19 bp, Homo sapiens
agttctgggagcggcggga

SEQ ID NO: 3 - DNA, 21 bp, Homo sapiens
cagagtcattggcgctatgga

SEQ ID NO: 4 - DNA, 17 bp, Homo sapiens
cgagctgggggcggata

SEQ ID NO: 5 - DNA, 186 bp, Homo sapiens
ggacaaagaaggggatgaccagagtcattggcgctatggaggcgacccgccctggccccgggtgtccccagcctgcgcgggccgcttccagtccccggtggatatccgcccccagctcgccgccttctgcccggccctgcgccccctggaactcctgggcttccagctcccgccgctcccagaact

SEQ ID NO: 6 - DNA, 200 bp, Homo sapiens
cagagtcattggcgctatggaggcgacccgccctggccccgggtgtccccagcctgcgcgggccgcttccagtccccggtggatatccgcccccagctcgcagagtcattggcgctatggaggcgacccgccctggccccgggtgtccccagcctgcgcgggccgcttccagtccccggtggatatccgcccccagctcg